(19) United States
(12) Patent Application Publication
(10) Pub. No.: US 2026/0047920 A1
(43) Pub. Date: Feb. 19, 2026

(54) ROTATOR CUFF IMPLANTS, SYSTEMS AND METHODS OF USING THE SAME

(71) Applicant: Orthopedic Surgical Specialist and Rehabilitation, PLLC, Kingston, NY (US)

(72) Inventor: Adam SOYER, Kingston, NY (US)

(73) Assignee: Orthopedic Surgical Specialist and Rehabilitation, PLLC, Kingston, NY (US)

(21) Appl. No.: 19/304,309

(22) Filed: Aug. 19, 2025

Related U.S. Application Data
(60) Provisional application No. 63/684,808, filed on Aug. 19, 2024.

(51) Int. Cl. A61F 2/08; A61L 27/34; A61L 27/52

(52) U.S. Cl. CPC A61F 2/0811; A61L 27/34; A61L 27/52; A61F 2002/0817; A61F 2002/087; A61F 2002/0888; A61F 2220/0016

(57) ABSTRACT

An implant, system, and method used for inserting a tendon repair implant system into a patient's humerus. The implant system includes an implant and at least one staple. The implant includes a body, a first arm extending away from the body on a first end of the implant, and a second arm extending away from the body between the first end and a second end of the implant. The implant may also include a hydrogel coating with a gradient of human growth factors positioned on at least the bone contacting surfaces. The implant is positioned on the proximal humerus and greater tuberosity with the two arms of the implant spaced apart forming a passageway for receiving the rotator cuff tendon.

### CROSS-REFERENCE TO RELATED APPLICATION

This application claims priority benefit under 35 U.S.C. § 119(e) of U.S. Provisional Application No. 63/684,808, filed Aug. 19, 2024 and entitled BIO CUFF-LINK, which is incorporated herein by reference in its entirety.

### FIELD OF THE INVENTION

The present invention relates generally to general surgery, orthopedic implants used for repairing a tendon in a joint, such as shoulder implants. More specifically, but not exclusively, the present invention relates to rotator cuff tendon repair, as well as methods for using the same.

### BACKGROUND OF THE INVENTION

Rotator cuff repair is the second most common soft tissue repair procedure done by orthopedic surgeons. By 2017, 83% of rotator cuff tears were arthroscopic procedures and 99% were performed on an outpatient basis. The incidence of rotator cuff repair was 165 per 100,000 population. Re-repair in the 50-64-year-old age group was as high as 423 per 100,000. Recent data would suggest that over 460,000 rotator cuff surgeries are performed each year and as the population continues to age, the demand for rotator cuff repairs is expected to increase reaching an estimate of 570,000 procedures by 2023.

The rotator cuff is a series of four muscle/tendon units that enable universal joint function of the shoulder. Anatomically, the four muscle/tendon units take origin from the scapula (shoulder blade). The attachment of each tendon (defined: structure that attaches muscle to bone) is to a specific point on the proximal humerus known as tuberosities (or prominences). The supraspinatus tendon thickness, at its attachment site, varies with age. The average tendon thickness is approximately 5 mm. Rotator cuff tendons generally tear in a consistent pattern. For a number of reasons, the rotator cuff tendon tears directly from its bony attachment on a prominence known as the greater tuberosity (GT). The rotator cuff tendon may then retract towards its muscular attachment on the scapula (or shoulder blade). Once torn, the rotator cuff tendon undergoes physiologic changes. The greater tuberosity also undergoes physiological changes that negatively impact rotator cuff tendon healing to bone.

The most common techniques for rotator cuff repair include using a combination of sutures and suture anchors to restore the rotator cuff tendon footprint to the greater tuberosity.

The limitations of these techniques are multifactorial, including: (1) the size and pattern of the rotator cuff tear, (2) difficulty achieving stable fixation of the tendon to bone, (3) reliant on the quality of the soft tissue supporting suture fixation (patient age, fatty atrophy), and (4) reliant on the integrity of the tendon/bone interface (enthesis) to enable healing of the tendon to bone (osteopenia/osteoporosis, prior surgical procedures).

The limitations of present surgical techniques are contributory to the high incidence of rotator cuff re-tears requiring revision surgery particularly with larger tears. When the rotator cuff tears, “the damaged interface lacks sufficient endogenous signal cues to regenerate functional tissue.” In essence, when the rotator cuff tendon tears from its native footprint on bone (GT), the process of healing and repair becomes disorganized and suboptimal. As a result, tissue regeneration strategies have been developed utilizing growth factors, stem cells and soft tissue augmentation procedures with tissue transplants (allograft, xenograft, autograft and decellularized structures), collagen matrices and synthetic reconstructive grafts.

There are four distinct regions of the tendon/bone interface, including: ligament/tendon zone 1, non-mineralized fiber cartilage zone 2, mineralized fibrocartilage zone 3, and bone zone 4. Zone 1 is mostly composed of collagen type I. Zone 2 mostly collagen to with high levels of pericellular collagen III. Zone 3 is mainly composed of collagen II with significant amounts of collagen X.

The rotator cuff tendon healing takes place in three stages including: inflammatory, reparative, and remodeling. In the inflammatory phase, Vascular Endothelial Growth Factor (“VGEF”) stimulates blood vessel formation in the repair tissue. In the reparative phase (week 1 to week 8), there is active proliferation of fibroblasts (FGF-Fibroblast Growth Factor) and differentiation of stem cells. TGF-beta 1 expression is seen in adult rotator cuff tears with a peak at day 10. Platelet derived growth factor (“PDGF”) has been linked to collagen formation. Additionally, bone morphogenic protein (“BMP”) was shown to be actively involved in the tissue remodeling phase. Growth factors are felt to be expressed in consistent patterns throughout the stages of rotator cuff healing.

Therefore, what is needed is an improved rotator cuff tendon repair implant that restores the natural rotator cuff tendon footprint for anatomic rotator cuff tendon repair.

### SUMMARY OF THE INVENTION

Aspects of the present invention provide glenoid implants for anatomic shoulder arthroplasties. The present invention also provides for methods for using the glenoid implants.

In one aspect, provided herein is an implant that includes a body, a first arm extending away from the body on a first end of the implant, and a second arm extending away from the body between the first end and a second end of the implant.

In another aspect, provided herein is a tendon repair implant system, including an implant and at least one staple for engaging at least a portion of the implant.

In yet another aspect, provided herein is a surgical method for inserting a tendon repair implant system, including obtaining the tendon repair implant system and preparing a proximal humerus and greater tuberosity for receiving the tendon repair implant system. The method may also include inserting an implant of the tendon repair implant system onto the proximal humerus and the greater tuberosity and inserting at least one staple of the tendon repair implant system through the implant.

These, and other objects, features and advantages of this invention will become apparent from the following detailed description of the various aspects of the invention taken in conjunction with the accompanying drawings.

### BRIEF DESCRIPTION OF DRAWINGS

The accompanying drawings, which are incorporated in and constitute a part of the specification, illustrate embodiments of the invention and together with the detailed description herein, serve to explain the principles of the invention. The drawings are only for purposes of illustrating preferred embodiments and are not to be construed as limiting the invention. It is emphasized that, in accordance with the standard practice in the industry, various features are not drawn to scale. In fact, the dimensions of the various features may be arbitrarily increased or reduced for clarity of discussion. The foregoing and other objects, features and advantages of the invention are apparent from the following detailed description taken in conjunction with the accompanying drawings in which:

FIG. 1 is a first perspective view of an embodiment of a rotator cuff tendon repair implant, in accordance with an aspect of the present disclosure;

FIG. 2 is a second perspective view of the implant of FIG. 1, in accordance with an aspect of the present disclosure;

FIG. 3 is a first side view of the implant of FIG. 1, in accordance with an aspect of the present disclosure;

FIG. 4 is a second side view of the implant of FIG. 1, in accordance with an aspect of the present disclosure;

FIG. 5 is a first end view of the implant of FIG. 1, in accordance with an aspect of the present disclosure;

FIG. 6 is a second end view of the implant of FIG. 1, in accordance with an aspect of the present disclosure;

FIG. 7 is a top view of the implant of FIG. 1, in accordance with an aspect of the present disclosure;

FIG. 8 is a bottom view of the implant of FIG. 1, in accordance with an aspect of the present disclosure;

FIG. 9 is a first perspective view of the implant of FIG. 1 with a coating on at least portions of the implant, in accordance with an aspect of the present disclosure;

FIG. 10 is a second perspective view of the implant of FIG. 9, in accordance with an aspect of the present disclosure;

FIG. 11 is a first perspective view of a staple for use with the implant of FIG. 1, in accordance with the present disclosure;

FIG. 12 is a second perspective view of the staple of FIG. 11, in accordance with an aspect of the present disclosure;

FIG. 13 is a top view of the staple of FIG. 11, in accordance with an aspect of the present disclosure;

FIG. 14 is a bottom view of the staple of FIG. 11, in accordance with an aspect of the present disclosure;

FIG. 15 is a first end view of the staple of FIG. 11, in accordance with an aspect of the present disclosure;

FIG. 16 is a second end view of the staple of FIG. 11, in accordance with an aspect of the present disclosure;

FIG. 17 is a first side view of the staple of FIG. 11, in accordance with an aspect of the present disclosure;

FIG. 18 is a second side view of the staple of FIG. 11, in accordance with an aspect of the present disclosure;

FIG. 19 is a first perspective view of an implant system with the implant of FIG. 1 and two staples of FIG. 11 engaging the implant, in accordance with an aspect of the present disclosure;

FIG. 20 is a second perspective view of the implant system of FIG. 19, in accordance with an aspect of the present disclosure;

FIG. 21 is a first perspective view of a portion of the humerus illustrating a tendon tear, in accordance with an aspect of the present disclosure;

FIG. 22 is a first perspective view of the humerus of FIG. 21 with an insertion guide, in accordance with an aspect of the present disclosure;

FIG. 23 is a first perspective view of the humerus of FIG. 22 with a punch, in accordance with an aspect of the present disclosure;

FIG. 24 is a first perspective view of the humerus of FIG. 23 with two sutures inserted through the tendon tear, in accordance with an aspect of the present disclosure;

FIG. 25 is a first perspective view of the humerus of FIG. 24 with the implant of FIG. 1 being inserted over the sutures, in accordance with an aspect of the present disclosure;

FIG. 26 is a first perspective view of the humerus of FIG. 25 with the implant positioned on the humerus and engaging a tendon, in accordance with an aspect of the present disclosure;

FIG. 27 is a second perspective view of the humerus of FIG. 26, in accordance with an aspect of the present disclosure;

FIG. 28 is a first perspective view of the humerus of FIG. 26 with the staples of FIG. 11 inserted through the implant of FIG. 1, in accordance with an aspect of the present disclosure;

FIG. 29 is a second perspective view of the humerus of FIG. 28, in accordance with an aspect of the present disclosure; and

FIG. 30 is a perspective view of a portion of the coating, in accordance with an aspect of the present disclosure.

### DETAILED DESCRIPTION FOR CARRYING OUT THE INVENTION

Generally stated, disclosed herein are rotator cuff tendon repair implants. Further, surgical methods for using the rotator cuff tendon repair implants are discussed.

In this detailed description and the following claims, the words proximal, distal, anterior, posterior, medial, lateral, superior and inferior are defined by their standard usage for indicating a particular part of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure.

As used herein, the word “exemplary” or “illustrative” means “serving as an example, instance, or illustration.” Any implementation described herein as “exemplary” or “illustrative” is not necessarily to be construed as preferred or advantageous over other implementations. Moreover, in the present description, the terms “upper,” “lower,” “left,” “rear,” “right,” “front,” “vertical,” “horizontal,” and derivatives thereof shall relate to the invention as oriented in the first figure of each embodiment.

Similarly, positions or directions may be used herein with reference to anatomical structures or surfaces. For example, as the current implants, devices, systems and methods are described herein with reference to use with the bones of the shoulder, the bones of the shoulder and upper arm may be used to describe the surfaces, positions, directions or orientations of the implants, devices, systems and methods. Further, the implants, devices, systems and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the implants, devices, systems and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the implants, devices, systems and methods, and the aspects, components, features and the like thereof, described herein with respect to the right shoulder may be mirrored so that they likewise function with the left shoulder and vice versa. Further, the implants, devices, systems and methods, and the aspects, components, features and the like thereof, disclosed herein are described with respect to the shoulder for brevity purposes, but it should be understood that the implants, devices, systems and methods may be used with other bones of the body having similar structures, for example the lower extremity, and more specifically, with the bones of the ankle, knee, hip, foot, and leg.

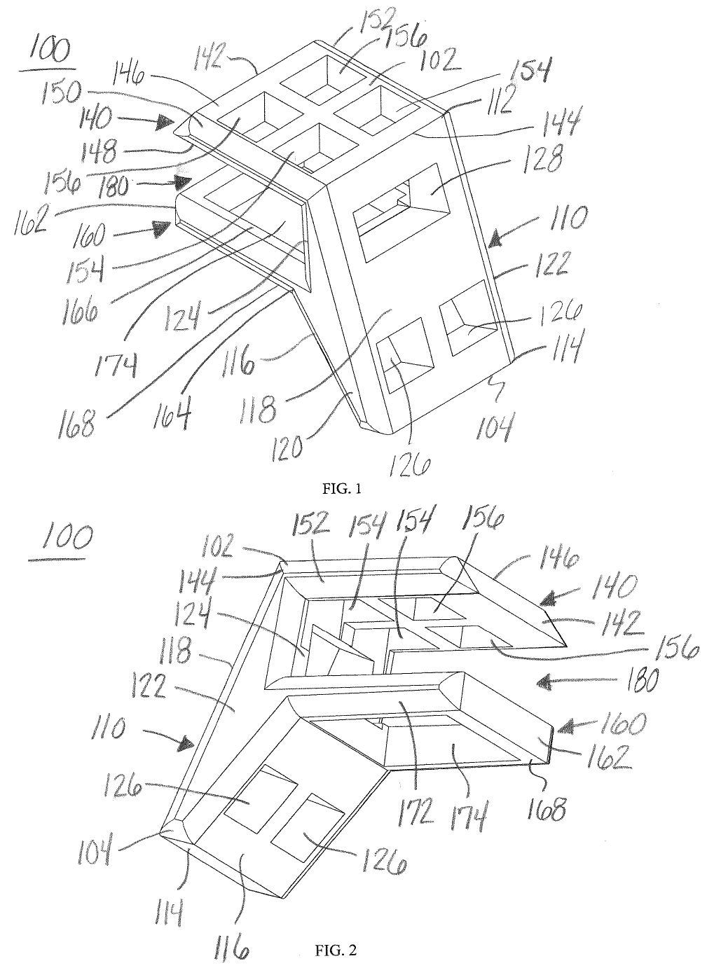

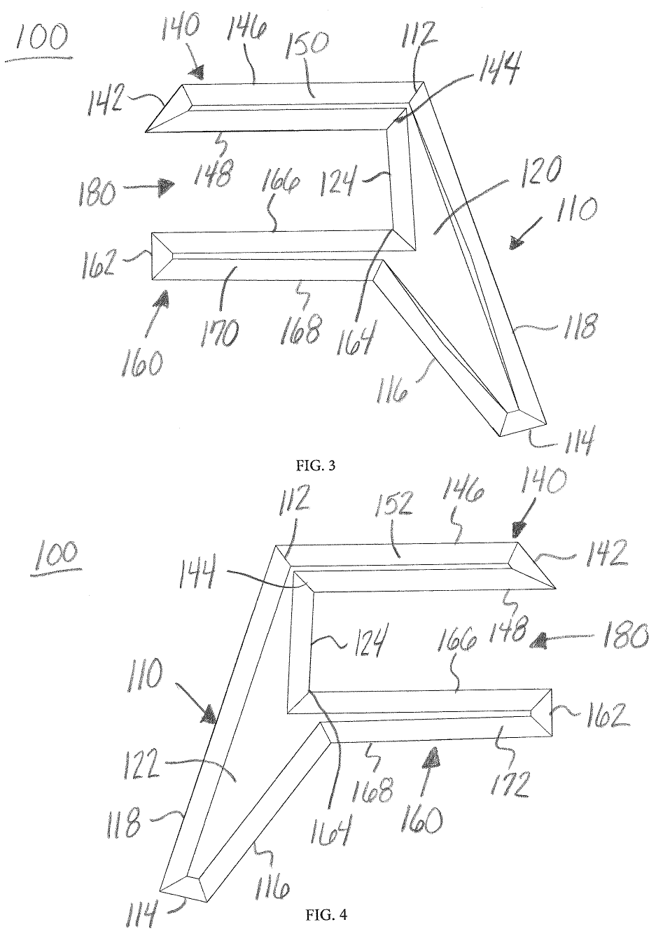

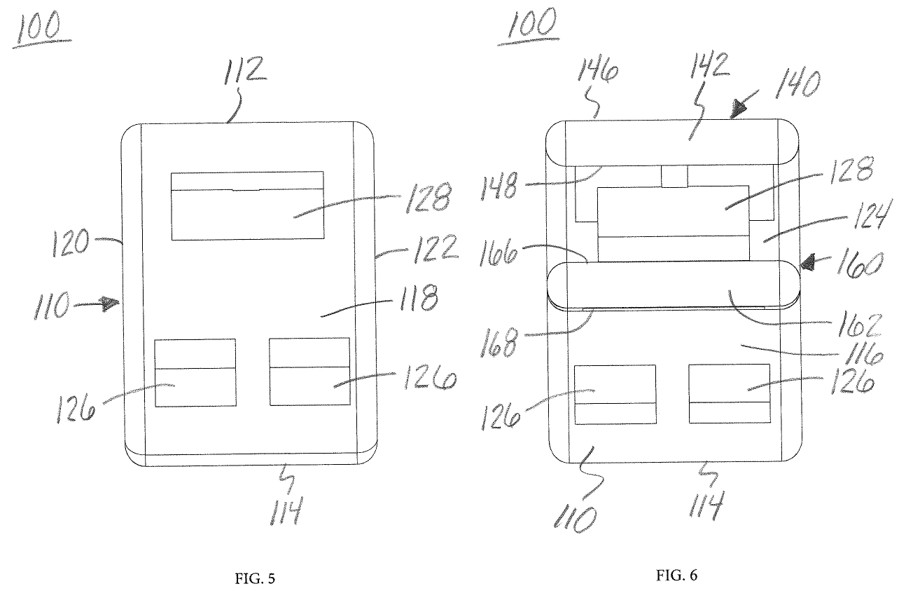

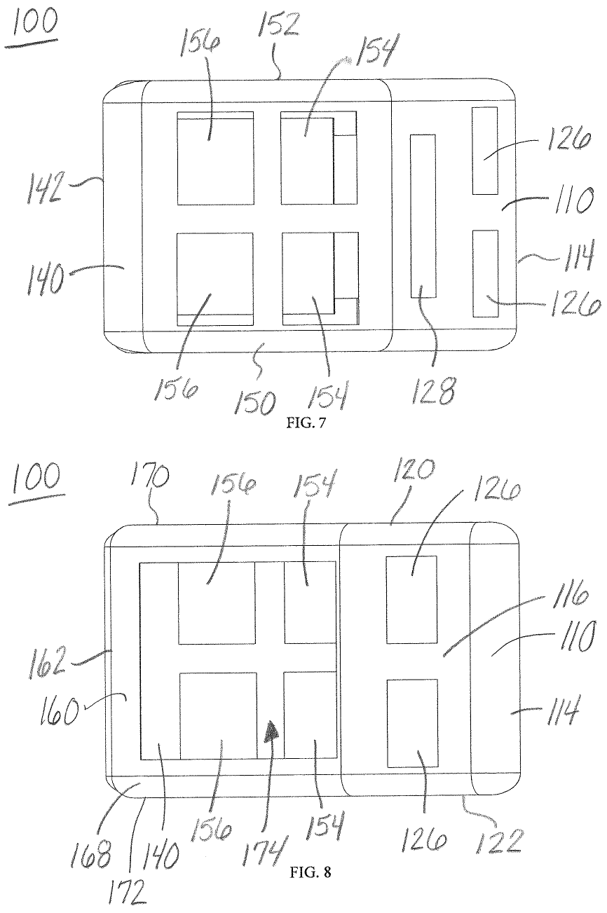

Referring to the drawings, wherein like reference numerals are used to indicate like or analogous components throughout the several views, and with particular reference to FIGS. 1-10, there is illustrated an embodiment of a rotator cuff tendon repair implant 100. The implant 100 includes a first end or proximal end 102 and a second end or distal end 104. The first end 102 is opposite the second end 104. The implant 100 includes a first portion or body 110, a first arm 140, and a second arm 160. The arms 140, 160 extend away from a medial side of the body 110. The first arm 140 may be, for example, coupled to and extending from a first end 112 of the body 110. The second arm 160 may be, for example, coupled to and extending from an interior surface 124 of the body 110 at a position intermediate the first end 112 and the second end 114. The first arm 140 may be spaced apart from the second arm 160 forming a passageway 180 between the first and second arms 140, 160 for receiving a tendon, for example, a rotator cuff tendon, when the implant 100 is inserted into a patient. The passageway 180 may be, for example, sized and shaped to receive the tendon, which may be inserted between the first and second arms 140, 160 and extend into the interior surface 124. The first arm 140 may be spaced apart from the second arm 160 along an interior surface 124 of the body 110.

With continued reference to FIGS. 1-10, the body 110 includes a first end 112, a second end 114 opposite the first end 112, a medial side 116, a lateral side 118 opposite the medial side 116, an anterior side 120, and a posterior side 122 opposite the anterior side 120. The medial side 116 and the lateral side 118 may diverge from each other as the sides 116, 118 extend from the second end 114 toward the first end 112. The body 110 may have, for example, a triangular shape. The body 110 may further include a pair of openings 126 extending through the body 110 from the medial side 116 to the lateral side 118. The pair of openings 126 may be positioned near the second or inferior end 114 of the body 110. The pair of openings 126 may be, for example, evenly spaced apart from each other between the anterior side 120 and the posterior side 122. The pair of openings 126 may be, for example, sized and shaped to receive a staple 200, as discussed in greater detail below. The pair of openings 126 may be, for example, angled relative to the first arm 140 and the second arm 160 as the openings 126 extend between the lateral side 118 to the medial side 116. Each of the openings 126 may have, for example, a square shape. The openings 126 may be, for example, positioned adjacent to each other between the anterior side 120 and the posterior side 122.

As shown in FIGS. 1-10, the body 110 also includes a window 128 extending through the body 110 from the medial side 116 to the lateral side 118. The window 128 may be positioned near the first or superior end 112 of the body 110. The window 128 may be, for example, angled relative to the first arm 140 and the second arm 160 as the window 128 extends between the lateral side 118 and the medial side 116. The window 128 may extend through the body 110 and be positioned between the first arm 140 and the second arm 160. The window 128 may be angled from the lateral side 118 of the body 110 towards the second arm 160. The window 128 may have, for example, a rectangular shape. The window 128 may be positioned superior to the pair of openings 126 on the body 110.

Also shown in FIGS. 1-10, the first arm 140 may include a first end or medial end 142 and a second end or lateral end 144 opposite the first end 142. The first arm 140 also includes a superior end or exterior surface 146 opposite an inferior end or interior surface 148.

The second end 144 of the first arm 140 is coupled to a first end 112 of the body 110. The body 110 extends away from the second end 144 of the first arm 140 at an angle. The first end 142 may be, for example, tapered or angled between the superior end 146 and the inferior end 148. The superior end 146 may have a first length extending between the lateral side 118 of the body 110 to the first end 142 of the first arm 140 and the inferior end 148 may have a second length extending between the lateral side 118 of the body 110 to the first end 142 of the first arm 140. The first length being shorter than the second length, such that the inferior end 148 of the first arm 140 extends beyond the superior end 146 of the first arm 140 at the first end 142 of the first arm 140.

With continued reference to FIGS. 1, 2, 7 and 8, the first arm 140 also including a first pair of openings 154 and a second pair of openings 156. The first pair of openings 154 may be positioned near the second end 144 of the first arm 140. The first pair of openings 154 may include, for example, two openings 154 positioned adjacent to each other between the first side 150 and the second side 152. The first pair of openings 154 may be, for example, evenly spaced apart from each other between the first side 150 and the second side 152. The first pair of openings 154 may be, for example, sized and shaped to receive a staple 200, as discussed in greater detail below. The first pair of openings 154 may, for example, extend perpendicularly through the first arm 140 from the superior end 146 to the inferior end 148. Each of the openings 154 may have, for example, a square shape. It is also contemplated that the openings 154 may have, for example, another polygonal shape.

The second pair of openings 156 may be positioned near the first end 142 of the first arm 140. The second pair of openings 156 may include, for example, two openings 156 positioned adjacent to each other between the first side 150 and the second side 152. The second pair of openings 156 may be, for example, evenly spaced apart from each other between the first side 150 and the second side 152. The second pair of openings 156 may be, for example, sized and shaped to receive a staple 200, as discussed in greater detail below. The second pair of openings 156 may, for example, extend perpendicularly through the first arm 140 from the superior end 146 to the inferior end 148. Each of the openings 156 may have, for example, a square shape. It is also contemplated that the openings 156 may have, for example, another polygonal shape. The first pair of openings 154 may be positioned adjacent to and aligned with the second pair of openings 156. For example, the first pair of openings 154 and the second pair of openings 156 may be positioned to form, for example, a grid pattern. The grid pattern may provide for structural stability and evenly distribute the forces across the tendon repair site. The surface of the first pair of openings 154 on the second end 144 may align with the interior surface 124 of the body 110.

The second arm 160 may also include a first end or medial end 162 opposite a second end or lateral end 164, a superior end 166 opposite an inferior end 168, and a first side or anterior side 170 opposite a second side or posterior side 172. The second end 164 of the second arm 160 is coupled to a medial side 116 of the body 110. The body 110 is angled as it extends away from the second arm 160 in both directions. The first end 162 may be, for example, flat or planar as it extends between the superior end 166 and inferior end 168 of the second arm 160. The superior end 166 may have a first length extending between the interior surface 124 of the body 110 to the first end 162 of the second arm 160 and the inferior end 168 may have a second length extending between the interior surface 124 of the body 110 to the first end 162 of the second arm 160. The first length of the second arm 160 may be the same as the second length of the second arm 160, such that the inferior end 168 of the second arm 160 extends to the same length as the superior end 166 of the second arm 160. The second arm 160 may also include a through hole 174 extending through the second arm 160 from the superior end 166 to the inferior end 168. The through hole 174 may be larger than each of the pairs of openings 154, 156. The side of the through hole 174 on the anterior side 170 of the second arm 160 may be, for example, aligned with the sides of the openings 154, 156 on the anterior side 150 of the first arm 140. In addition, the side of the through hole 174 on the posterior side 172 of the second arm 160 may be, for example, aligned with the sies of the openings 154, 156 on the posterior side 152 of the first arm 140. Further, the through hole 174 may be spaced apart from the interior surface 124 of the body 110.

The dimensions of the implant 100 may be selected based on the size of the patient's humerus and rotator cuff tendon. In one embodiment, the body 110 of the implant 100 may be 12.5 mm along the anterior side 120 and the posterior side 122 between the first end 112 and the second end 114, as well as along the superior end 112 and the inferior end 114 between the anterior side 120 and the posterior side 122. The width of the second end 114 between the medial side 116 and the lateral side 118 may be 2.25 mm. The openings 126 may have a dimension of 4 mm by 3.5 mm and the window 128 may have a dimension of 7.5 mm by 3.5 mm. The first arm 140 may have dimensions of 10 mm on the inferior end 148 and a height of 2.25 mm on the medial end 142. The openings 154, 156 of the first arm 140 may have dimensions of 3.5 mm by 4.25 mm. The second arm 160 may have dimensions of 10 mm on the superior end 166 and the inferior end 168 and a height of 2.25 mm on the medial end 162. The through hole 174 of the second arm 160 may have dimensions of 9 mm by 9 mm. In addition, the inferior end 148 of the first arm 140 may be separated from the superior end 166 of the second arm 160 by 4.5 mm. The implant 100 may also have an angle of approximately 110° between the inferior end 168 of the second arm 160 and the medial side 116 of the body 110.

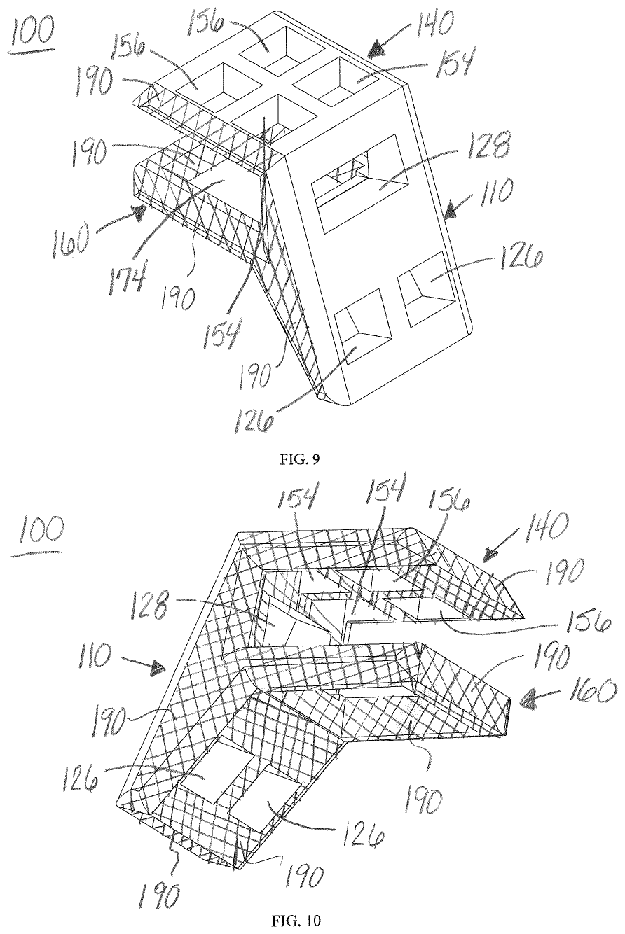

Regarding FIGS. 9-10, the implant 100 may include a coating 190 positioned on at least the bone contacting and/or tendon contacting surfaces. For example, the coating 190 may be formed on the interior end 148 of the first arm 140, and the superior end 166, the inferior end 168, the lateral end 164, the anterior side 170, and the posterior side 172 of the second arm 160. In addition, the coating 190 may be formed on the medial end 142, the anterior side 150, and the posterior side 152 of the first arm 140 and the medial side 116, the anterior side 120, and the posterior side 122 of the body 110. Further, the coating 190 may be formed on the interior surface of the through hole 174 of the second arm 160. Alternatively, the coating 190 may be positioned on all surfaces of the implant 100.

The coating 190 may be, for example, a hydrogel for biologic healing. The hydrogel may be a water-based, porous, cross-linked polymer which provides a biologic delivery system for delivery of key factors in promoting tendon to bone healing or enthesis. The hydrogel polymer may be impregnated with key factors. The key factors or human growth factors may include fibroblast growth factor (FGF), transforming growth factor beta subunit 1 (TGF-B1), platelet derived growth factor—BB (PDGF-BB), and bone morphogenic protein subunits 7 and 12 (BMP 7/12). The coating 190 may have a gradient of the human growth factors. When the human growth factors are introduced in a gradient, the factors can be released in a staggered array mimicking the stages of tendon to bone healing. These key factors or biologic factors are human cytokines or growth factors that have been identified as promoting staged healing in tendon to bone repairs. The hydrogel of the coating 190 may be a hyaluronic acid hydrogel. The hydrogel or polymer reinforces weakened or torn tendon tissue allowing for broader distribution of the forces for footprint reattachment.

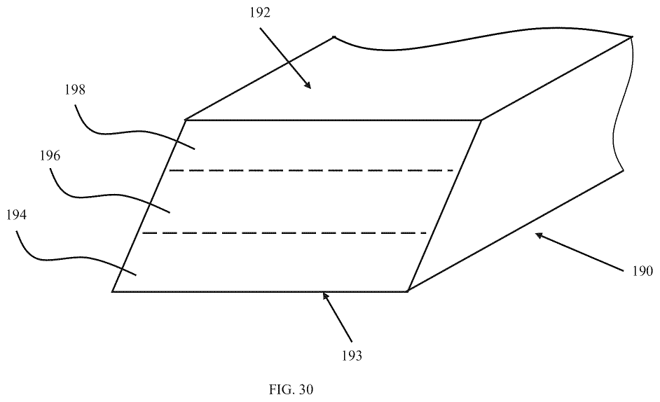

In an embodiment, the coating 190 may be a multi-layer hydrogel with gradients for delivery of the factors over time. For example, the coating 190 may be a hyaluronic acid hydrogel providing growth factor and/or drug delivery with a three-level gradient for delivery of factors over time. The three-level coating 190 may be formed into a sheet for placing on all surfaces of the implant 100. The first layer 194 of the three-level coating 190 would release the factors and/or medication over a period of two weeks, then the second layer 196 would release the factors and/or medication over the next two-week period, and finally, the third layer 198 would release the factors and/or medication over the following two-week period, as shown in FIG. 30. Thus, the factors and/or medication would be released from the coating 190 over a six-week period. The three layer gradient with staged release of growth factors and/or doxycycline based on proximity to the enthesis 314, 316. The three-layers may include an enthesis layer 194, a middle layer 196, and a superior layer 198 between a superior surface 192 and an inferior surface 193 of the coating sheet 190. The enthesis layer 194 may consist of doxycycline, BMP subunit 7, and PDGF as initial release factors. The middle layer 196 may consist of PDGF and GFG subunit 2 as the second release factors. Finally, the superior layer 198 may consist of PDGF and TGF beta 1 as the third release factors. In an embodiment, the concentrations of medication and/or growth factors may be 0.02 mg/dL of doxycycline, 500 ng/dl of BMP subunit 7, 5 mcg/dL of PDGF, 5 mcg/dL of FGF subunit, and 400 ng/ml of TGF-beta 1.

The implant 100 may be, for example, formed by 3D printing or other known biocompatible implant formation methods. The implant 100 may be made of, for example, a porous polypropylene filament or like material. The porous polypropylene filament may be, for example, a 2.85 mm porous filament. The implant 100 may be, for example, a one piece, non-modular, singular integral or monolithic piece (i.e., of one-piece construction). Alternatively, in some embodiments, the implant 100 may be formed from more than one component that are coupled (i.e., rigidly coupled) together to form the implant 100.

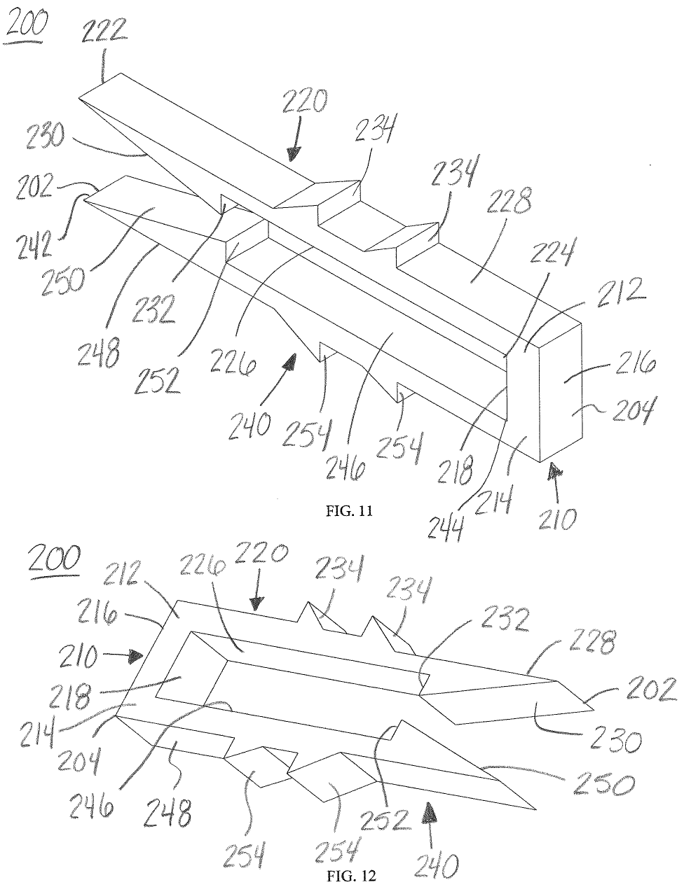

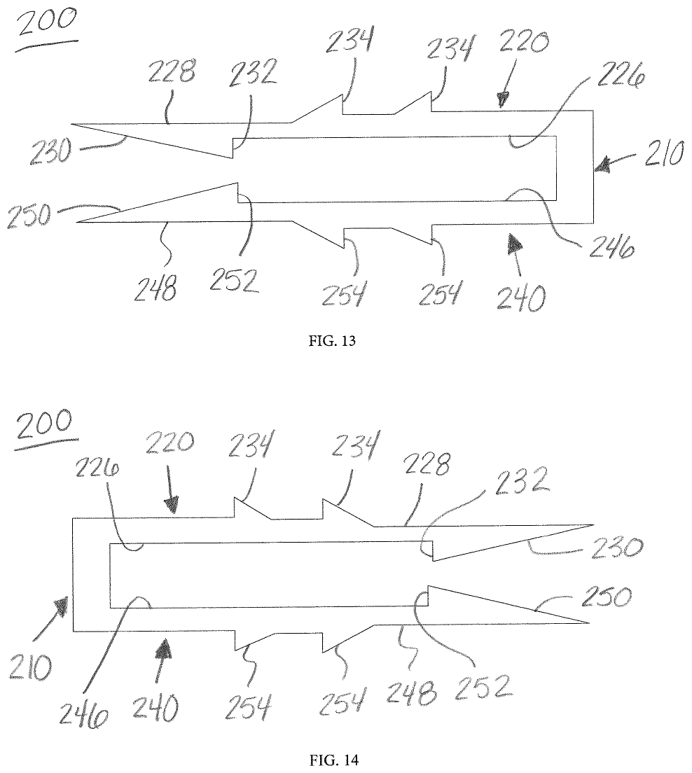

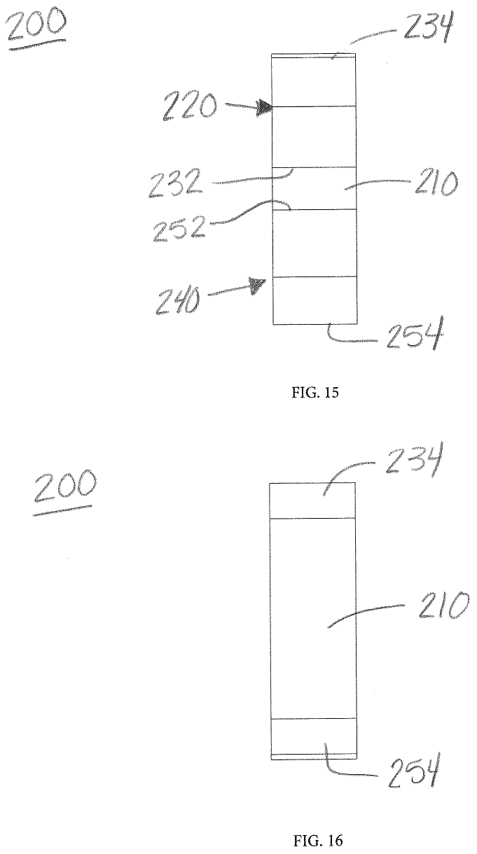

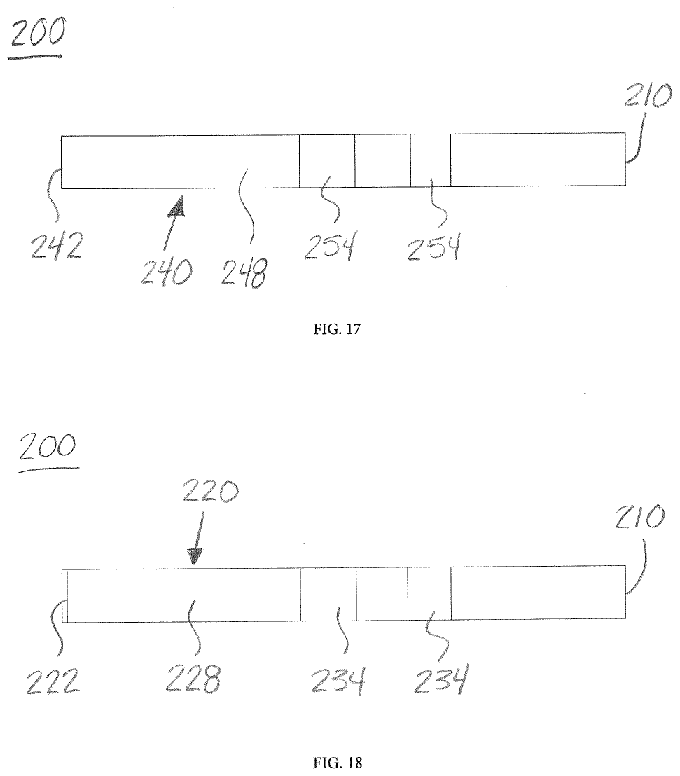

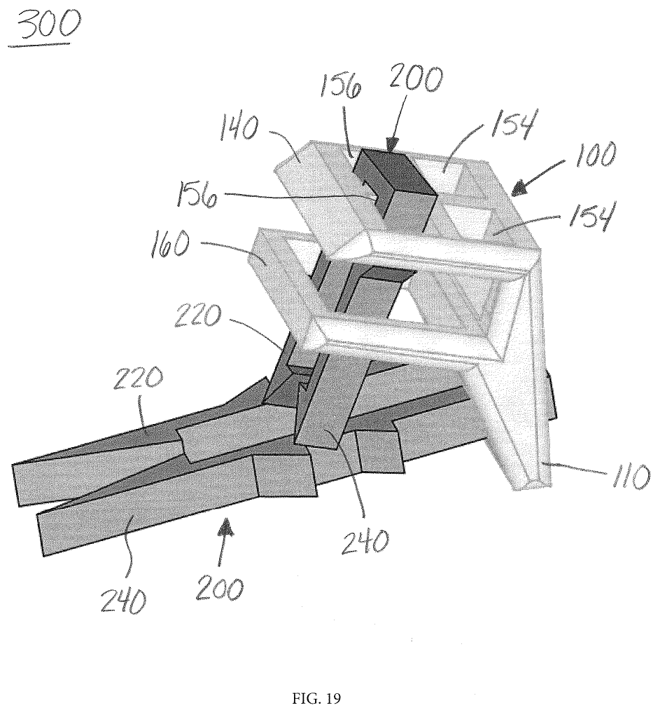

Referring now to FIGS. 11-18, the staple 200 is shown. The staple 200 has a first end 202 and a second end 204 opposite the first end 202. The staple 200 has a body 210, a first extension 220 extending away from a first end 212 of the body 210, and a second extension 240 extending away from a second end 214 of the body 210. The first extension 220 being a mirror image of the second extension 240. The body 210 also includes an exterior surface 216 and an interior surface 218 extending between the first end 212 and the second end 214. The interior surface 218 being opposite the exterior surface 216. The first and second extensions 220, 240 extend away from the interior surface 218 of the body 210.

With continued reference to FIGS. 11-18, the first extension 220 includes a first end or insertion end 222 opposite a second end or coupling end 224. The coupling end 224 couples the first extension 220 to the body 210. The insertion end 222 has a planar or flat surface on the exterior surface 228 and a tapered or angled surface on the interior surface 226. The angled surface on the interior surface 226 creates a first engagement member 230. The first extension 220 also includes a tooth 232 positioned at the end of the first engagement member 230. The tooth 232 extends away from the interior surface 226 of the first extension 220. The interior surface 226 angles from the insertion end 222 to the tooth 232. The exterior surface 228 of the first extension 220 may also include at least one protrusion 234 extending away from the exterior surface 228. The at least one protrusion 234 may be, for example, two protrusions 234 positioned between the first end 222 and the second end 224. More specifically, the at least one protrusion 234 is positioned between the tooth 232 of the first engagement member 230 and the second end 224. The tooth 232 and at least one protrusion 234 are configured or sized and shaped to engage a tendon and secure the tendon to a patient's bone.

The second extension 240 includes a first end or insertion end 242 opposite a second end or coupling end 244, as shown in FIGS. 11-18. The coupling end 244 couples the second extension 240 to the body 210. The insertion end 242 has a planar or flat surface on the exterior surface 248 and a tapered or angled surface on the interior surface 246. The angled surface on the interior surface 246 creates a second engagement member 250. The second extension 240 also includes a tooth 252 positioned at the end of the second engagement member 250. The tooth 252 extends away from the interior surface 246 of the second extension 240. The interior surface 246 angles from the insertion end 242 to the tooth 252. The exterior surface 248 of the second extension 240 may also include at least one protrusion 254 extending away from the exterior surface 248. The at least one protrusion 254 may be, for example, two protrusions 234 positioned between the first end 242 and the second end 244. More specifically, the at least one protrusion 254 is positioned between the tooth 252 of the second engagement member 250 and the second end 244. The tooth 252 and at least one protrusion 254 are configured or sized and shaped to engage a tendon and secure the tendon to a patient's bone.

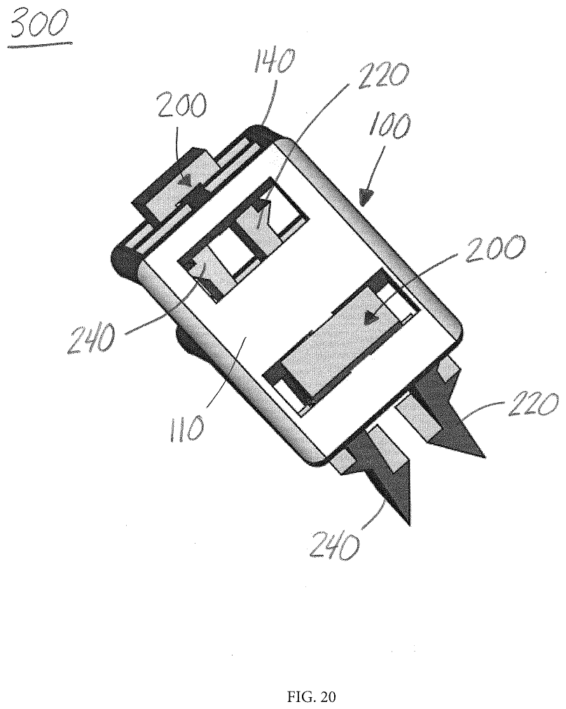

A tendon repair system 300 is shown in FIGS. 19 and 20. The tendon repair system 300 includes the implant 100 and at least one staple 200. As shown, the tendon repair system 300 includes an implant 100 and two staples 200. The implant 100 includes a first staple 200 inserted through the openings 126 of the body 110 and a second staple 200 inserted through the openings 156 in the first arm 140. The first or lateral staple 200 may be inserted through the implant 100 to secure the implant 100 to a humerus. The second or superior staple 200 may be inserted through the implant 100 and tendon and into the humerus. The second staple 200 may also pass through the through hole 174 of the second arm 160.

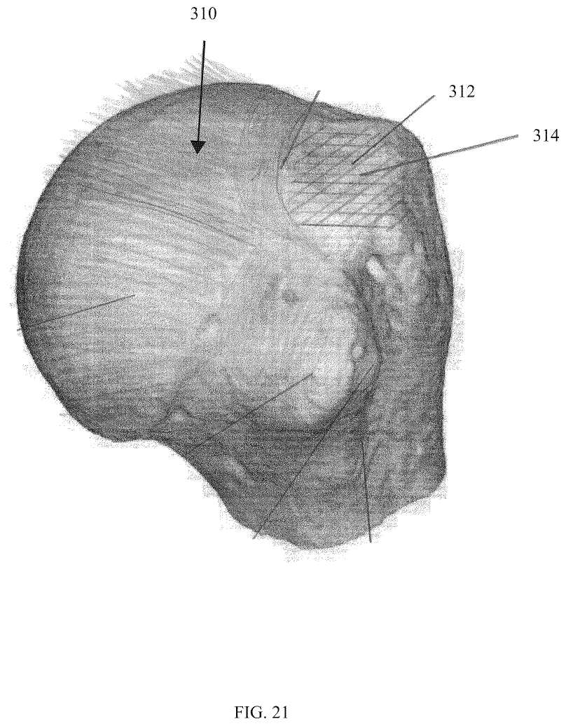

Referring now to FIGS. 21-29, the method for inserting the implant 100 using the staples 200 is shown. The surgical procedure may be performed by either open or arthroscopic procedures. As shown in FIG. 21, the method includes exposing the proximal humerus 310 of the injured arm to expose where the rotator cuff tendon 316 is torn from the greater tuberosity 312. Once the greater tuberosity is exposed, the footprint of the native tendon attachment section 314 will also be exposed. The surfaces of the proximal humerus 310 and greater tuberosity 312 may then be prepared to recess the implant 100 satisfactorily to enable tendon to bone healing.

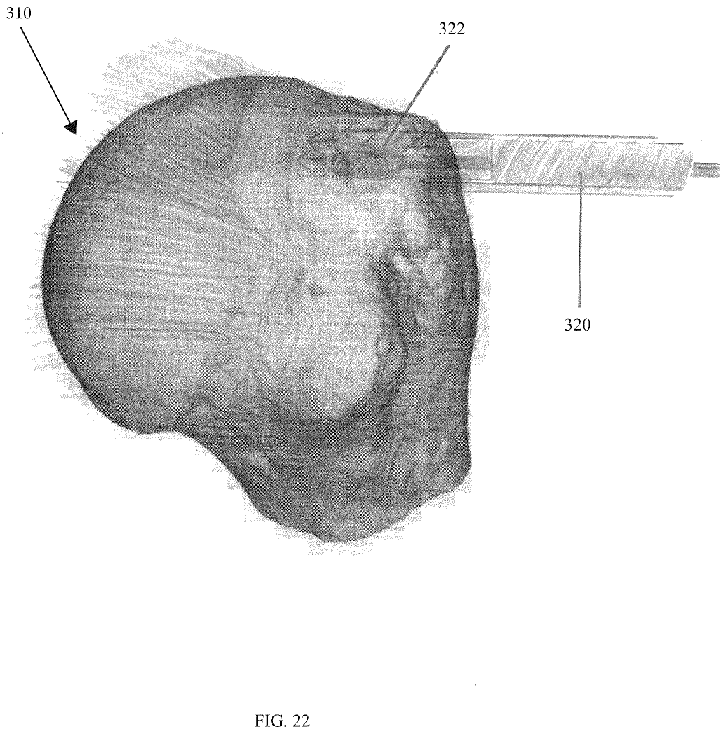

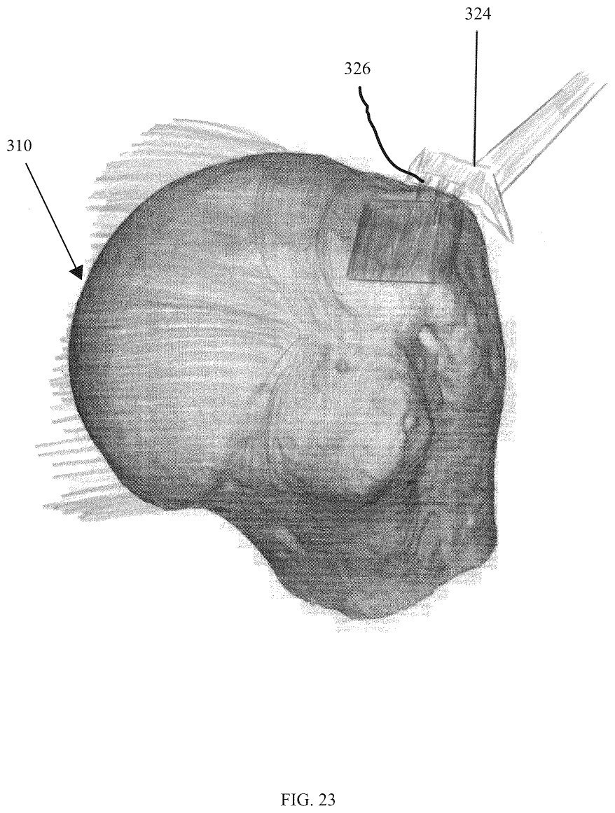

Initially, the method includes introduction of an insertion guide 320 on the lateral aspect of the greater tuberosity 312 to allow for resurfacing of the greater tuberosity 312 through a lateral portal approach, as shown in FIG. 22. The greater tuberosity 312 may be, for example, resurfaced 2 mm. Then, a barrel burr 322 may be inserted through the insertion guide 320 to further flatten the greater tuberosity 312, as also shown in FIG. 22. The barrel burr 322 may be inserted in a plunge fashion from lateral to medial to the edge of the articular surface. Multiple sweeping side passes may be performed to plane the surface of the greater tuberosity 312 flat. In one embodiment, a 3 mm barrel burr 322 may be used taking, for example, three passes side to side sweeping passes with the barrel burr 322 to flatten the tuberosity 312. After flattening the tuberosity 312, a punch 324 may be introduced through a superior accessory portal onto the flattened greater tuberosity 312, as shown in FIG. 23. The punch 324 may be, for example, shaped the same or similar to the final implant 100. The punch 324 may have a multi-pronged surface including barbs 326 that pierce the subchondral bone into the bone marrow and peripheral ridge to recess the implant 100 into the humerus 310. The punch 324 may include, for example, three barbs 326. When the punch 324 is used, the extrusion of bone marrow releases stem cells and growth factors to the surface of the bone to complement or promote biologic healing. In addition, the punch 324 indents the periphery of the greater tuberosity 312 to allow the implant 100 to recess and remain flush with the prepared bone surface.

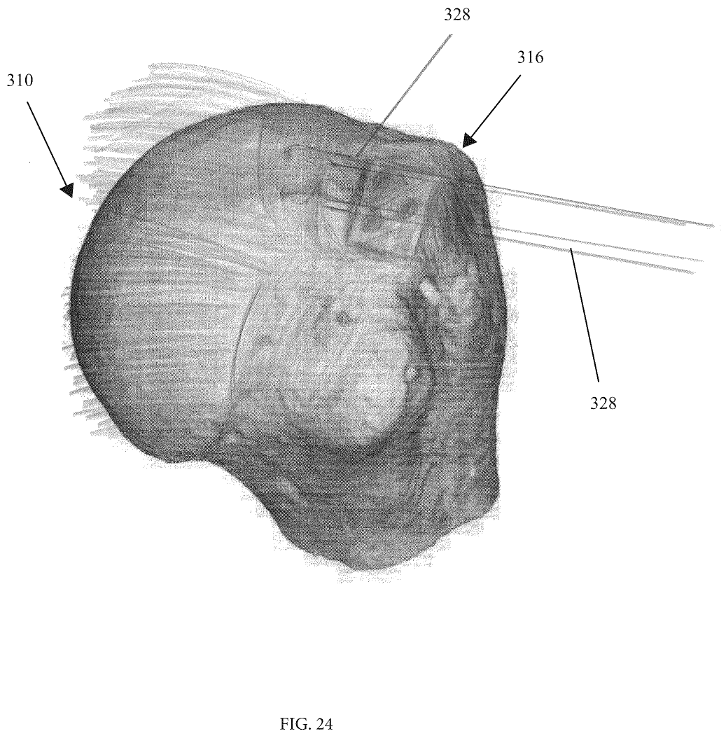

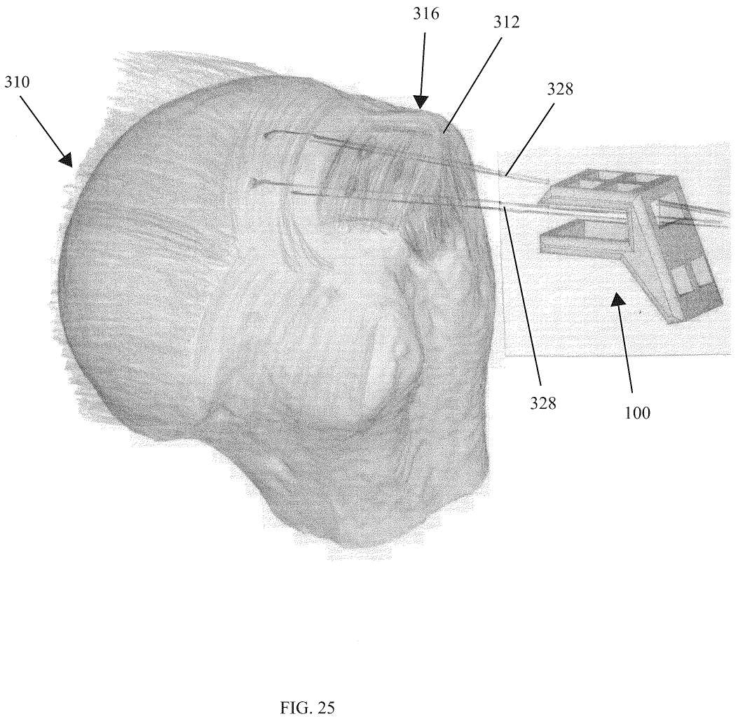

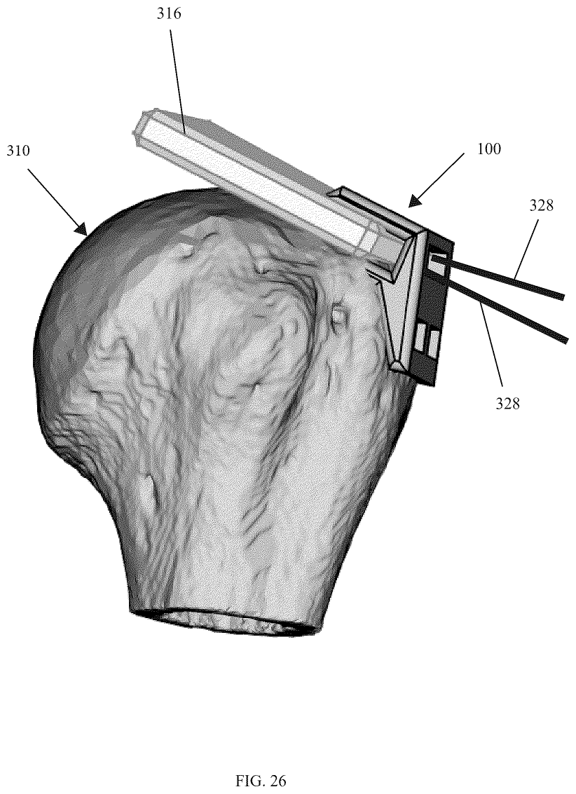

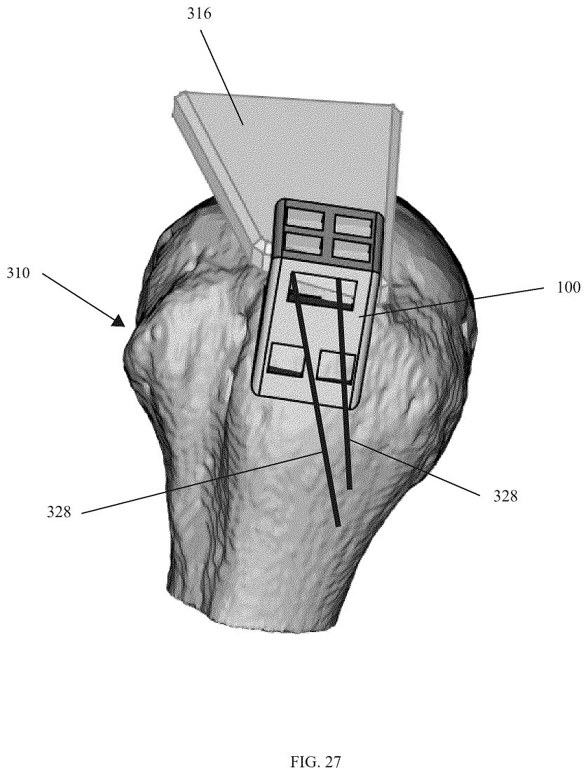

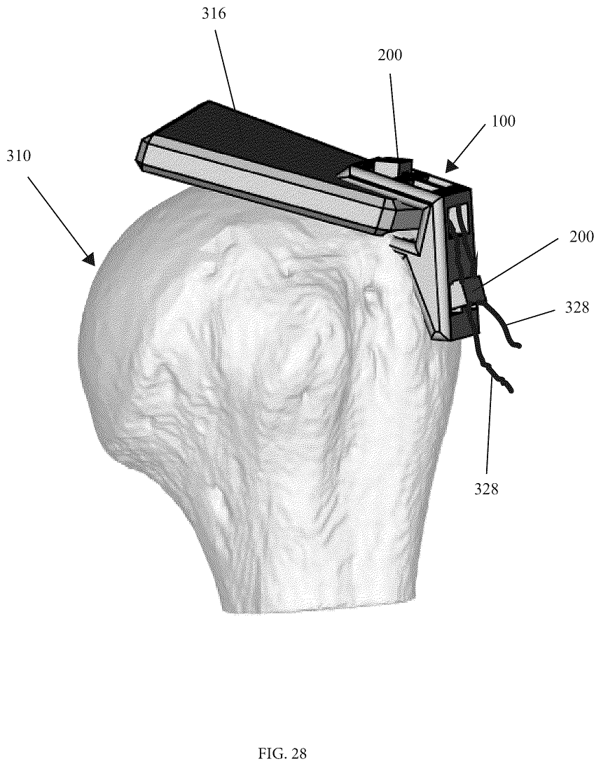

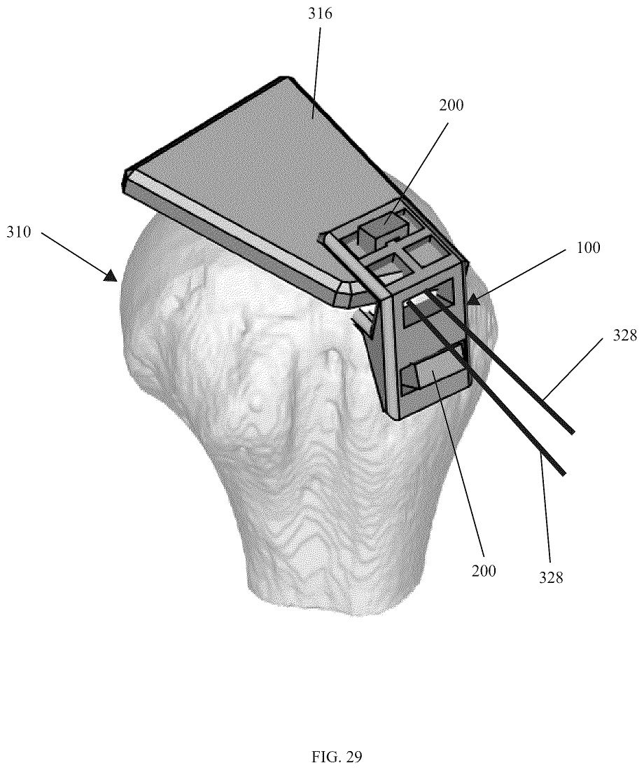

After the bone 310 is prepared, two sutures 328 are passed through the torn rotator cuff tendon 316 through the lateral portal, as shown in FIG. 24. Next, the implant 100 may be passed over the sutures 328 inserted through the torn rotator cuff tendon 316 as the implant 100 is secured to the proximal humerus 310, as shown in FIG. 25. The implant 100 is then seated on the greater tuberosity 312 and the lateral aspect of the proximal humerus 310 with the sutures 328 passed through the window 128 in the implant 100, as shown in FIGS. 26 and 27. As the implant 100 is moved onto the bone 310, the sutures 328 are used to guide the tendon 316 into the passageway 180. The rotator cuff tendon 316 may be positioned within the tendon passageway 180. Then, a staple punch may be introduced from a lateral and superior position to create pilot holes for the staples 200. The staple punch may be, for example, an arthroscopic staple punch. The staple punch may be used first to create pilot holes in the lateral side of the bone 310 and then to create pilot holes in the superior surface of the bone 310. The staples may be, for example, polylactic acid absorbable staples. Further, as shown in FIGS. 28 and 29, the implant 100 is secure to the bone 310 by staples 200. A first staple 200 is inserted through the body 110 of the implant 100 and into the humerus 310 on a lateral side. The inserted sutures 328 are docked over the staple 200 with the implant 100 being permanently secure to the bone 310 with the rotator cuff tendon 316 advanced far laterally for additional stability of the repair. Then, a second staple 200 is passed through a pair of openings 154, 156 of the grid on the superior surface of the implant 100 securing the rotator cuff tendon 316 directly to the bone 310 and restoring the rotator cuff footprint. In addition, the second staple 200 assists with securing the implant 100 to the bone 310.

Where a patient has a larger rotator cuff tear, more than one implant 100 can be stacked behind the first implant 100 to address the larger tear requiring fixation. Any additional implants 100 may be inserted onto the humerus 310 using the same method as described in greater detail above. It is also contemplated that where stacking or linking multiple implants are used to secure the rotator cuff tendon 316, the staple 200 may be positioned in one opening 154, 156 in the first implant 100 and in one opening 154, 156 in a second implant 100.

After at least one implant 100 is secured to the bone 310 with the staples 200 and the sutures 328, then the procedure can be complete and the incision closed. Once the procedure is complete, the implant 100 including coatings 190 will start to act as biologic cues to stimulate healing of the tendon to bone interface.

As may be recognized by those of ordinary skill in the art based on the teachings herein, numerous changes and modifications may be made to the above-described and other embodiments of the present disclosure without departing from the scope of the disclosure. The components of the implants, devices, and/or systems as disclosed in the specification, including the accompanying abstract and drawings, may be replaced by alternative component(s) or feature(s), such as those disclosed in another embodiment, which serve the same, equivalent or similar purpose as known by those skilled in the art to achieve the same, equivalent or similar results by such alternative component(s) or feature(s) to provide a similar function for the intended purpose. In addition, the implants, devices, and/or systems may include more or fewer components or features than the embodiments as described and illustrated herein. Accordingly, this detailed description of the currently preferred embodiments is to be taken in an illustrative, as opposed to limiting of the disclosure.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

Having thus described the preferred embodiments, the invention is now claimed to be:

1. An implant, comprising:
a body;
a first arm extending away from the body on a first end of the implant; and
a second arm extending away from the body between the first end and a second end of the implant.

2. The implant of claim 1, wherein the first arm is spaced apart from the second arm forming a passageway extending into the implant from a medial end of the implant towards a lateral end.

3. The implant of claim 1, wherein the body comprises:
a pair of openings extending through the body from a medial side to a lateral side, wherein the pair of openings are positioned near a second end of the implant.

4. The implant of claim 3, wherein the body further comprises:
a window extending through the body from a medial side to a lateral side, wherein the window is positioned near a first end of the implant.

5. The implant of claim 1, wherein the first arm is positioned parallel to the second arm, and wherein the body extends away from a second end of each of the first arm and the second arm at an angle.

6. The implant of claim 1, wherein the first arm comprises:
at least one pair of openings extending through the first arm from a superior end to an inferior end.

7. The implant of claim 6, wherein the at least one pair of openings comprises:
a first pair of openings positioned near a medial side of the first arm; and
a second pair of openings positioned near a lateral side of the first arm.

8. The implant of claim 7, wherein the openings of the first pair of openings and the second pair of openings are positioned in a grid pattern.

9. The implant of claim 1, wherein the second arm comprises:
a through hole extending through the second arm from a superior end to an inferior end.

10. The implant of claim 1, further comprises:
a hydrogel coating covering at least bone contacting surfaces of the implant, wherein the hydrogel coating comprises:
a first layer coupled to the implant;
a second layer; and
a third layer, wherein the second layer is positioned between the first layer and the third layer.

11. A tendon repair implant system, comprising:
an implant; and
at least one staple for engaging at least a portion of the implant.

12. The system of claim 11, wherein the implant comprises:
a body;
a first arm extending away from the body on a first end of the implant; an
a second arm extending away from the body between the first end and a second end of the implant.

13. The system of claim 12, wherein the body comprises:
a pair of openings extending through the body from a medial side to a lateral side, wherein the pair of openings are positioned near a second end of the implant, wherein a first staple of the at least one staple is inserted through the pair of openings.

14. The system of claim 13, wherein the first arm comprises:
a first pair of openings positioned near a medial side of the first arm; and
a second pair of openings positioned near a lateral side of the first arm;
wherein a second staple of the at least one staple is inserted through at least one of the first pair of openings and the second pair of openings.

15. The system of claim 12, further comprises:
at least one suture extending through a window of the body of the implant.

16. A method for inserting a tendon repair implant system, comprising:
obtaining the tendon repair implant system;
preparing a proximal humerus and greater tuberosity for receiving the tendon repair implant system;
inserting an implant of the tendon repair implant system onto the proximal humerus and the greater tuberosity; and
inserting at least one staple of the tendon repair implant system through the implant.

17. The method of claim 16, wherein inserting at least one staple of the tendon repair implant system through the implant comprises:
inserting a first staple of the at least one staple into a lateral side of the proximal humerus; and
inserting a second staple of the at least one staple into a superior side of the proximal humerus.

18. The method of claim 16, further comprising:
inserting at least one suture through a torn rotator cuff tendon before inserting the implant.

19. The method of claim 16, further comprising:
inserting at least one second implant posterior to the inserted implant.

20. The method of claim 16, wherein the implant includes a coating on at least bone contacting surfaces.
